(19) United States
(12) Patent Application Publication
(10) Pub. No.: US 2024/0425769 A1
(43) Pub. Date: Dec. 26, 2024

(54) MULTIPLE FLUIDIZED BED OR SPOUTED BED REACTORS FOR PLASTICS PYROLYSIS

(71) Applicant: W.R. Grace & Co.-CONN., Columbia, MD (US)

(72) Inventors: Wu-Cheng CHENG, Columbia, MD (US); Guang YUAN, Clarksville, MD (US); Robert Hibbard HARDING, Woodstock, MD (US); Abubacker SIDDIEQ, Chennai (IN); Anapagaddi RAVIKIRAN, Chennai (IN)

(73) Assignee: W.R. Grace & Co.-CONN., Columbia, MD (US)

(21) Appl. No.: 18/699,261

(22) PCT Filed: Oct. 5, 2022
(86) PCT No.: PCT/US2022/045801

§ 371 Date: Apr. 5, 2024

Related U.S. Application Data
(60) Provisional application No. 63/252,929, filed on Oct. 6, 2021.

(51) Int. Cl. C10G 55/06; B01D 21/26; B01J 6/00; C10B 49/22; C10B 53/07; C10B 57/02; C10B 57/06; C10B 57/14; C10B 57/16

(52) U.S. Cl. CPC C10G 55/06; B01D 21/267; B01J 6/008; C10B 49/22; C10B 53/07; C10B 57/02; C10B 57/06; C10B 57/14; C10B 57/16; C10G 2300/1003; C10G 2400/20; C10G 2400/22; C10G 2400/30

(57) ABSTRACT

A system for converting plastic includes a catalyst regenerator, a feeder containing plastic feedstock, a first conical spouted bed reactor stage in fluid communication with the catalyst regenerator and in fluid communication with the feeder, and a second conical spouted bed reactor stage in fluid communication with the first conical spouted bed reactor stage.

### CROSS REFERENCE TO RELATED APPLICATIONS

This application claims the benefit of priority to U.S. Provisional Patent Application No. 63/252,929 filed Oct. 6, 2021, which is hereby incorporated by reference, in its entirety for any and all purposes.

### FIELD

The present technology is generally related to the conversion of plastics to lower molecular weight hydrocarbon products. Specifically, the technology is related to the use of conical spouted bed reactors in series to convert plastic feedstock into olefin and aromatic products through pyrolysis.

### BACKGROUND

Plastic waste is a growing concern due to the material's lack of biodegradability. However, many plastic materials are excellent candidates for creating a circular economy—a paradigm in which 100% of waste is able to be recycled or used to produce feedstock for other useful materials.

Pyrolysis of waste plastics in a fluidized bed or spouted bed reactor has been suggested as a potential route to convert the waste plastics to a liquid fuel or chemical feedstock, which can be used to make recycled plastics. Most of the published work has relied on a fixed fluidized bed or fixed spouted bed, where millimeter-size plastic is injected continuously into a fixed bed of catalyst. While catalytic pyrolysis has been extensively studied, there remains a need to develop more efficient catalytic pyrolysis methods that maximize of the yield of desirable products, such as light olefins and aromatic compounds, and minimize the yield of undesirable products, such as methane and ethane. In particular, propylene is a particular light olefin in high demand as it is used in many of the world's largest and fastest growing synthetic materials and thermoplastics.

This disclosure provides a system and a process that is capable of producing olefins, such as propylene, and aromatic products with high selectivity from plastic feedstock.

### SUMMARY

Pyrolysis of plastic within fluidized bed reactors and spouted bed reactors has been suggested as a potential pathway to produce olefins and aromatics. However, there are serious difficulties with such a proposal that, before now, have not been adequately solved. Pyrolysis of millimeter size plastics particles in a fluidized bed or spouted bed reactor occurs on a time scales of hundreds of seconds. In a fluidized bed or spouted bed reactor, the catalyst flow is highly back-mixed, with a non-uniform catalyst residence time distribution. Due to constant circulation of catalyst between the reactor and a catalyst regenerator and the non-uniform catalyst and plastic residence time distribution within the reactor at any given time, there will be entrainment of unconverted plastics entering the regenerator. The present invention solves this problem, by having two or more fluidized beds or spouted beds in series, which greatly narrows the residence time distribution of the catalyst and plastics and decreases the fraction of unconverted plastics, entrained by the catalyst, circulating from the reactor to regenerator.

In a first aspect, a system for converting plastic into lower molecular weight products is herein disclosed, the system including a catalyst regenerator, a feeder containing plastic feedstock, a first conical spouted bed reactor stage in fluid communication with the catalyst regenerator and in fluid communication with the feeder, and a second conical spouted bed reactor stage in fluid communication with the first conical spouted bed reactor stage. In some embodiments, a third conical spouted bed reactor stage in fluid communication with the second conical spouted bed reactor stage is used.

In some embodiments, the first and second conical spouted bed reactor stages are contained within a single reactor vessel. In some of these embodiments, the first and second conical spouted bed reactor stages are at least partially separated by baffles. In some of these embodiments, the baffles define an opening at the top, bottom, or one or more sides of the first reactor stage.

In other embodiments, each conical spouted bed reactor stage is contained in a separate reactor vessel. In some of these embodiments, the vessels are positioned at different elevations.

In some embodiments, the material within a reactor stage including catalyst and unreacted plastic feedstock is able to pass from the first reactor stage to the second via a pipe or other passage. In some of these embodiments, the pipe or passage is aerated such that the flow of catalyst and unreacted feedstock is pneumatically driven from the first reactor stage to the second.

In some embodiments, the first conical spouted bed reactor stage is configured to receive catalyst from the catalyst regenerator. In some of these embodiments, the flow of catalyst from the catalyst regenerator to the first conical spouted bed reactor stage is adjustable in response to a temperature in the first conical spouted bed reactor stage falling below a predetermined temperature set point. In some embodiments, the second reactor stage is also in fluid communication with the catalyst regenerator and is configured to receive catalyst from the catalyst regenerator. In some of these embodiments, the flow of catalyst from the catalyst regenerator to the second conical spouted bed reactor stage is adjustable in response to a temperature in the second conical spouted bed reactor stage falling below a predetermined temperature set point. In some embodiments, a third reactor stage is also in fluid communication with the catalyst regenerator and is configured to receive catalyst from the catalyst regenerator. In some of these embodiments, the flow of catalyst from the catalyst regenerator to the third conical spouted bed reactor stage is adjustable in response to a temperature in the third conical spouted bed reactor stage falling below a predetermined temperature set point.

In some embodiments, the reactor stages include draft tubes, each tube extending from the bottom of the associated reactor stage toward the top of the reactor stage, where the draft tube includes a cylindrical tube having an outer diameter smaller than the inner diameter of the bottom of the reactor stage and at least one opening extending upward from the bottom of the draft tube.

In some embodiments, the reactor stages include a confiner, each confiner extending from the top of the associated reactor stage toward the bottom of the reactor stage, the confiner comprising a cylindrical tube having an outer diameter smaller than the inner diameter of the top of the reactor stage.

In some embodiments, the first conical spouted bed reactor stage operates at a temperature of about 300° C. to about 650° C., or from about 450° C. to about 600° C., or from about 480° C. to about 550° C. In some embodiments, the second conical spouted bed reactor stage operates at a temperature of about 300° C. to about 650° C., or from about 450° C. to about 600° C., or from about 480° C. to about 550° C.

In some embodiments, the system further includes a gas feed system in fluid communication with the first conical spouted bed reactor stage and the second conical spouted bed reactor stage, and the gas feed system is configured to feed a motive gas to the first conical spouted bed reactor stage and the second conical spouted bed reactor stage. In some of these embodiments, the motive gas contains less than 1.0 wt. % oxygen or, more preferably, less than 0.1 wt. % oxygen.

In some embodiments, the system includes a set of separation cyclones in fluid communication with the first conical spouted bed reactor stage and the second conical spouted bed reactor stage.

In a second aspect, a method of producing hydrocarbon product from plastic is disclosed, where the method includes feeding a plastic feedstock and motive gas into a first conical spouted bed reactor stage containing a catalyst to produce a first product vapor and a first residual plastic, separating at least a portion of the first product vapor from the motive gas and the first residual plastic to produce a first product stream comprising the first product vapor, feeding the first residual plastic from the first conical spouted bed reactor stage and motive gas into a second conical spouted bed reactor stage containing a catalyst to produce a second product vapor and a second residual plastic, and separating at least a portion of the second product vapor from the motive gas and the second residual plastic to produce a second product stream comprising the second product vapor.

In some embodiments, the method includes transferring at least a portion of the catalyst from the first conical spouted bed reactor stage to the second conical spouted bed reactor stage. In some embodiments, the method includes transferring at least of portion of the catalyst from the second conical spouted bed reactor stage to a regenerator. In some embodiments, the method includes feeding catalyst from the regenerator into the first conical spouted bed reactor stage. In some embodiments, the method includes feeding catalyst from the regenerator into the second conical spouted bed reactor stage. In some embodiments the transfer of the portion of the catalyst from the first conical spouted bed reactor stage to the second conical spouted bed reactor stage is at least partly driven by a flow of motive gas. In some embodiments the transfer of the portion of the catalyst from the second conical spouted bed reactor stage to the regenerator is at least partly driven by a flow of motive gas.

In some embodiments the first conical spouted bed reactor stage has a temperature from about 300° C. to about 650° C., or from about 450° C. to about 600° C., or from about 480° C. to about 550° C. In some of these embodiments, the temperature of the first conical spouted bed reactor stage is controlled in part through feeding hot catalyst into the first conical spouted bed reactor stage from the regenerator. In some embodiments the second conical spouted bed reactor stage has a temperature from about 300° C. to about 650° C., or from about 450° C. to about 600° C., or from about 480° C. to about 550° C. In some of these embodiments, the temperature of the second conical spouted bed reactor stage is controlled in part through feeding hot catalyst into the second conical spouted bed reactor stage from the regenerator.

In some embodiments, the plastic feedstock is first shredded to a nominal size of about 1 mm to about 20 mm, or preferably from about 8 mm to about 10 mm, prior to feeding into the first conical spouted bed reactor stage. In some embodiments, the method includes feeding the second residual plastic from the second conical spouted bed reactor stage and motive gas into a third conical spouted bed reactor stage containing catalyst to produce a third product vapor and a residue; and separating the third product vapor, motive gas, and residue to produce a third product stream comprising the third product vapor.

In some embodiments, the method includes directing the first product stream and the second product stream into a cyclone separator. In some of these embodiments, the first product stream and second product stream are combined before being directed into a cyclone separator. In some embodiments, the method includes collecting the first product stream and the second product stream into a separation vessel. In some embodiments, the plastic feedstock includes high density polyethylene, medium density polyethylene, low density polyethylene, polypropylene, polyethylene terephthalate, polyvinyl chloride, or a mixture of any two or more thereof.

In some embodiments, the first and second hydrocarbon products includes C¹-C¹² saturated hydrocarbons, C¹-C¹² unsaturated hydrocarbons, or a mixture of any two or more thereof, and wherein the first and second hydrocarbon products may be the same or different. In some embodiments, the hydrocarbon product include olefins, aromatic compounds, or a mixture of any two or more thereof.

In some embodiments, the method includes processing and refining one or more of the first hydrocarbon product, the second hydrocarbon product, the first plastic residue, or the second plastic residue in a steam cracker, a hydrocracker, a fluid catalytic cracker, a deep catalytic cracker, a high severity fluid catalytic cracker, a steam reformer, a liquid cracker gas plant, or aromatic recovery unit.

In some embodiments, the size of the first conical spouted bed reactor stage is the same as the size of the second conical spouted bed reactor stage. In some embodiments, the method is performed continuously. In some embodiments, the plastic feedstock includes a waste plastic. In some embodiments, separating the at least a portion of the first product vapor from the motive gas and the first residual plastic to produce a first product stream comprising the first product vapor occurs within the first conical spouted bed reactor stage.

In some embodiments, the first product stream is removed from the first conical spouted bed reactor stage immediately as it is formed. In some embodiments, separating the at least a portion of the second product vapor from the motive gas and the second residual plastic to produce a second product stream comprising the second product vapor occurs within the second conical spouted bed reactor stage. In some embodiments, the second product stream is removed from the second conical spouted bed reactor stage immediately as it is formed.

In some embodiments, the average gas phase residence time in the second conical spouted bed reactor stage is about 0.2 seconds to about 60 seconds, or preferably about 0.5 seconds to about 5 seconds. In some embodiments, the first conical spouted bed reactor stage and the first conical spouted bed reactor stage are operated in a fast pyrolysis regime. In some embodiments, the motive gas contains less than 1.0 wt. % oxygen or, more preferably, less than 0.1 wt. % oxygen.

### BRIEF DESCRIPTION OF THE DRAWINGS

FIG. 1 is a graph of fractional conversion of HDPE, LDPE, and PP at 500° C. and 550° C. as a function of time, according to the examples.

FIG. 2 is a graph of the residence time distribution of continuous flow in and out of a series of well-mixed reactors, according to the examples.

FIG. 3 is a graph of the sum of unconverted HDPE from each residence time interval in a series of well-mixed reactors at 550° C., according to the examples.

FIG. 4 is a graph of the sum of unconverted PP from each residence time interval in a series of well-mixed reactors at 550° C., according to the examples.

FIG. 5 is a schematic representation of two reactor vessels in series, according an illustrative embodiment.

FIG. 6 is a schematic of configurations of a single reactor vessel containing three reaction chambers, separated by baffles, where in the figure on the left, all chambers at the same elevation, and in the figure on the right, the chambers at decreasing elevation, according to various embodiments.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

### System for Converting Plastic Feedstock Into Valuable Hydrocarbon Products

Disclosed herein is a system for converting plastic feedstock into more valuable hydrocarbon feedstock, such as olefins and aromatics, in high yields. The objective of the system is to provide a continuous process for pyrolysis of plastics waste in a spouted bed reactor. The system herein disclosed features novel reactor design, comprising two or more spouted beds in series to continuously convert plastic to lower molecular weight products while continuously circulating catalyst between the reactors and a regenerator to burn off coke. It has been found that the use of a conical spouting bed reactor minimizes unconverted plastics being circulated from the reactor to the regenerator, increasing overall efficiency of the process. The use of multiple reactors in series also increases the uniformity of the plastics residence time within the system and substantially eliminates plastics bypass to the regenerator.

The system includes a catalyst regenerator, a feeder containing the plastic feedstock, at least two conical spouted bed reactor stages in fluid communication with each other, with the first of the reactor stages also being in fluid communication with the feeder. A conical spouted bed reactor stage includes a bottom, frustoconical portion and a cylindrical portion extending from the bottom portion. Inlets for the plastic feedstock and the catalyst are typically provided near the top of the reactor stage. At the bottom of the reactor stage an inlet for motive gas is provided.

While the present disclosure focuses on systems with two or three reactor stages, this is done for simplicity and brevity of the disclosure and should not be interpreted as limiting the system to only two or three reactor stages. In some of these embodiments, a fourth conical spouted bed reactor stage in fluid communication with the third conical spouted bed reactor stage is used. In some embodiments, a greater number of reactor stages is used. For example, five, six, seven, eight, nine, or ten reactor stages may be used.

During operation of the system, plastic feedstock is fed into a reactor stage containing catalyst in a catalyst bed. In some embodiments, catalyst is also fed into the reactor stage. In some embodiments, the catalyst is fed separately from the plastic feedstock. In other embodiments, the plastic feedstock and catalyst are co-fed.

Motive gas is fed into the reactor stage by a gas feed system. The flowing gas creates a cylindrical path, or spout, through the catalyst bed. Catalyst, entrained by the gas flowing through the spout, is propelled above the surface of the catalyst bed and settles back down in the shape of a fountain. The catalyst moves downward in the annular region back to the bottom of the conical bed, thus completing the cycle. The rapid circulation of the catalyst and reactants ensure good mixing in the reactor. The fountain is a region of low catalyst density, called the dilute phase, and the annulus is a region of high catalyst density, called the dense phase. In the absence of a draft tube, some of the gas flows around the spout and through the annular region. Unreacted plastic feedstock (or plastic feedstock particles that have not been fully converted into product) may pass from one reactor stage to a subsequent stage. If more than two stages are used, unreacted plastic feedstock within a reactor stage may flow into each subsequent stage until the final stage is reached. Catalyst may also flow from one stage to a subsequent stage. Operating the reactor stages in series has many benefits, including but not limited to increased conversion of feedstock to products, increased residence time of feedstock within the reactors, and reduced carryover of feedstock to the catalyst regenerator unit.

In some embodiments, in operation of the system, the transfer of unreacted plastic feedstock and catalyst from one reactor stage to the next may be facilitated by a pipe connecting the two reactor stages. The transfer of materials between the stages will partly be driven by the flow of gases flowing through the system such as the motive gas fed into each reactor stage. It is contemplated that the flow between the reactors may be further facilitated by positioning subsequent reactor stages at lower elevations than the preceding reactor stage so that the movement of material from one stage to the next may be at least partly driven by gravity. The pipe connecting the stages may also be aerated with an inert gas such as, for example, nitrogen, so that the transfer of the material is pneumatically driven. Aeration may be used when the reactor stages are positioned at different elevations or when the reactor stages are at the same elevation. Other means of facilitating transfer of material between the reactor stages, such as the use of an auger or similar physical means, are also contemplated.

In some embodiments, each reactor stage is contained in the same reactor vessel. In other embodiments, multiple stages are contained in a single reactor vessel. For example, in a system including three reactor stages, each reactor stage may be contained in single vessel. Alternatively, each reactor stage may be contained in a separate vessel. Or, alternatively, one reactor stage may be contained in a vessel separate from the other two stages (e.g. the first reactor stage is contained in a vessel separate from the vessel containing the second and third stages).

In embodiments in which multiple reactor stages are contained in a single vessel, baffles may be used to separate the reactor stages from one another. Baffles may be positioned to provide a passage between the reactor stages. For example, a baffle may be positioned between two stages such that a passage exists at the top of one of the stages, at the bottom of one of the stages, or at the side of one of the stages. Combinations are also possible and it should be understood that, due to the different positions of reactor stages within the reactor vessel, the top of one reactor stage may not correspond to the top of the next reactor stage.

As previously mentioned, a catalyst regenerator is used in the system. In operation, the catalyst used to facilitate the pyrolysis of the plastic feedstock may become deactivated through the buildup of coke. The catalyst is continuously cycled between the reactor stages and the regenerator. Within the regenerator, the catalyst is exposed to high temperatures and oxygen or air to burn off the coke buildup, thereby regenerating the catalyst. In some embodiments, the deactivated catalyst is exposed to air. The hot catalyst is then directed back to the reactor stages. Hot catalyst may be fed to any of the reactor stages. For example, hot catalyst may be fed to the first reactor stage or the second reactor stage or to each reactor stage.

The catalyst feed may be used to maintain the temperature within a reactor stage by providing heat needed for the pyrolysis of the plastic feedstock. Since the pyrolysis of the plastic feedstock is an endothermic reaction, additional heat input is required. This heat may be made up by the hot catalyst. In some embodiments, the catalyst flow rate to a reactor is adjustable within the system to keep the reactor stage at a predetermined temperature set point. For example, if the temperature within a reactor stage drops below a low temperature set point, the flow rate of hot catalyst from the regenerator to that reactor stage may be increased, causing the temperature in the reactor stage to increase. Conversely, if the temperature within a reactor stage climbs above a high temperature set point, the flow rate of hot catalyst from the regenerator to that reactor stage may be decreased, causing the temperature in the reactor stage to decrease.

In some embodiments, the reactor stages include draft tubes to direct the flow of motive gas and to induce enhanced mixing of the catalyst and the plastic feedstock. In some embodiments, the draft tube extends from the bottom of the reactor stage toward the top of the reactor stage. In some embodiments, the draft tube may be positioned concentrically with an inlet for the motive gas. The draft tube includes a cylindrical tube having an outer diameter smaller than the inner diameter of the bottom of the associated conical spouted bed reactor stage. In some embodiments, the ratio of the draft tube diameter to the motive gas inlet diameter is from about 1:1 to about 2:1. The draft tube may include at least one opening extending upward from the bottom of the draft tube through which catalyst material may pass through. In some embodiments, the draft tube is an open sided draft tube. In other embodiments, the draft tube is non-porous. The motive gas, flowing through the draft tube, creates a region of negative pressure, at the bottom of the tube, which pulls in catalyst from the annular region through the slot and propels it up the draft tube. Catalyst contained within the reactor stage can thereby become entrained by the motive gas, causing the material to mix. The draft tube directs the gas through the spout so that less gas travels through the annulus, as compared to the conventional spouted bed reactor. Thus, the minimum spouting velocity, in the presence of the draft tube, is much lower than in the absence of the draft tube.

In some embodiments, the reactor stages include confiners which extend from the top of the reactor stage toward the bottom. In some embodiments, the confiner is a cylindrical tube extending from the top of the reactor stage toward the bottom. The confiner may be placed concentrically with a plastic feedstock inlet at the top of the reactor. The confiner, which is closed at the top, redirects spouted catalyst downward. The confiner serves to reduce the volume available to gas and vapor phases in the reactor stage, causing the feedstock fed from the top of the reactor to more rapidly mix with the catalyst material spouted into the confiner volume. This results in more turbulent mixing of catalyst and plastic, which results in higher heat transfer, melting of the plastic feedstock, the molten particles of which become distributed on the catalyst particles.

The use of a conical spouted bed reactor allows the use of plastic feedstock that is of a much higher diameter than what is traditionally used. Those skilled in the art would expect the plastic feedstock to have been processed to have an average nominal particle size of 1 mm or less. Processing of plastic feedstock may include melting the plastic and cutting the extruded material into the desired size. In the present system presently disclosed, a particle size of between about 1 mm to 20 mm may be used. Preferably, the plastic feedstock has an average nominal particle size of between about 8 mm and 10 mm.

In some embodiments, the reactor stages are operated in a pyrolysis regime. In some embodiments, the reactor stages are operated in a “fast pyrolysis” regime, in which reactor stages are operated at pyrolysis temperatures and the gas phase has a residence time of one second or less. In some embodiments, the reactor stages operate at a temperature from about 300° C. to about 650° C., or more preferably from about 450° C. to about 600° C., or most preferably from about 480° C. to about 550° C. The reactor stages may all operate at the same temperature, or each reactor stage may operate at a different temperature depending on the conversion needs of the system or to adjust the selectivity of the products.

In some embodiments, the motive gas is an inert gas. In some embodiments, the motive gas is nitrogen, argon, steam, or a combination thereof. In some embodiments, the motive gas is less than 1.0 wt. % oxygen, or more preferably, less than 0.1 wt. % oxygen. In some embodiments, the motive gas is substantially free of oxygen.

To reduce degradation of the products, the system may include means for separating and collecting the products immediately as they are produced. The product vapors may be separated and collected from each reactor. This prevents the product vapors from traveling through each subsequent reactor stage. In some embodiments, the system also includes other equipment to process the hydrocarbon product as it is produced. In some embodiments, the system includes a set of cyclone separators in fluid communication with at least one of the reactor stages. In some of these embodiments, the set of cyclone separators is connected to each reactor stage so that a single set of cyclone separators may service the system. In other embodiments, each reactor stage has a separate set of cyclone separators. In other embodiments, the system includes at least one of a steam cracker, a hydrocracker, a fluid catalytic cracker, a deep catalytic cracker, a high severity fluid catalytic cracker, a steam reformer, a liquid cracker gas plant, or aromatic recovery unit.

### Process for Converting Plastic Feedstock Into Valuable Hydrocarbon Products

A second aspect, a method of producing valuable hydrocarbon products from plastics, is also disclosed herein. The method includes the steps of feeding a plastic feedstock and motive gas into a first conical spouted bed reactor stage containing a catalyst to produce a first product vapor and a first residual plastic, separating at least a portion of the first product vapor from the motive gas and the first residual plastic to produce a first product stream comprising the first product vapor, feeding the first residual plastic from the first conical spouted bed reactor stage into a second conical spouted bed reactor stage containing a catalyst to produce a second product vapor and a second residual plastic, and separating at least a portion of the second product vapor from the motive gas and the second residual plastic to produce a second product stream comprising the second product vapor. In some embodiments, the method also includes feeding the second residual plastic from the second conical spouted bed reactor stage into a third conical spouted bed reactor stage containing catalyst to produce a third product vapor and a third residual plastic, and separating the third product vapor, motive gas, and third residual plastic to produce a third product stream comprising the third product vapor. In some of these embodiments, the method also includes feeding the third residual plastic from the third conical spouted bed reactor stage into a fourth conical spouted bed reactor stage containing catalyst to produce a fourth product vapor and a residue, and separating the fourth product vapor, motive gas, and residue to produce a fourth product stream comprising the fourth product vapor. Additional reactor stages may be used if needed to achieve the desired overall conversion of the plastic feedstock.

The reactor stages used in the method may be housed in a single reactor vessel or may be distributed among multiple reactor vessels. For example, in an embodiment using three reactor stages, all three stages are contained in a single reactor vessel. In another embodiment using three reactor stages, each reactor stage is contained in a separate vessel. In another embodiment using three reactor stages, the first and second reactor stages are contained in a reactor vessel and the third stage is contained in a separate reactor vessel. In another embodiment using three reactor stages, the first is contained in a reactor vessel and the second and third stages are contained in a separate reactor vessel.

The method is applicable to several different plastic feedstocks. The feedstocks may include high density polyethylene, medium density polyethylene, low density polyethylene, polypropylene, polyethylene terephthalate, polyvinyl chloride, or a mixture of any two or more thereof. In some embodiments, the plastic feedstock is derived from plastic waste. In some embodiments, the plastic feedstock is primarily plastic waste.

The plastic feedstock is commonly pre-processed to achieve an average nominal particle size of between about 1 mm and about 20 mm, or more preferably between about 8 mm and about 10 mm. In some embodiments, the plastic feedstock has an average nominal particle of about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm.

The reactor stages used in this method may be operated in pyrolysis conditions or fast pyrolysis conditions. To achieve pyrolysis conditions, the temperature of the reactor stages is elevated to greater than about 300° C. and the amount of oxygen available to the system is limited. In some embodiments, the reactor stages operate at a temperature from about 300° C. to about 650° C. In some embodiments, the reactor stages operate at a temperature from about 450° C. to about 600° C. In some embodiments, the reactor stages operate at a temperature from about 480° C. to about 550° C.

The reactors in this method are operated in such a way that each reactor stage has an average gas phase residence time from about 0.2 seconds to about 60 seconds, or preferably about 0.5 seconds to about 5 seconds.

Each reactor stage contains a catalyst to facilitate the pyrolysis of the plastic feedstock. The ratio of mass of catalyst to mass of plastic in each stage varies by stage. In some stages, the ratio of mass of catalyst to mass of fuel ranges from about 5:1 to about 15:1, or more preferably from about 8:1 to about 10:1. In other stages, the ratio of mass of catalyst to mass of fuel ranges from about 15:1 to about 40:1, or more preferably from about 25:1 to about 35:1. The method may include transferring catalyst from one reactor to another reactor stage, thereby permitting the catalyst to flow through the reactor stages so that it may eventually travel to the regenerator. In some embodiments, the method includes the steps of transferring at least a portion of the catalyst from the first conical spouted bed reactor stage to the second conical spouted bed reactor stage. In other embodiments, the method includes the steps of transferring at least a portion of the catalyst from the second conical spouted bed reactor stage to a third conical spouted bed reactor stage. Or, stated generally, in some embodiments, the method includes the steps of transferring at least a portion of the catalyst from a given conical spouted bed reactor stage to the subsequent conical spouted bed reactor stage. The transfer of catalyst from one stage to the next may be driven by the flow of motive gas, the flow of pneumatic or aerating gas, gravity, or a combination thereof.

As the catalyst is used to process the plastic feedstock, the catalyst may become deactivated due to the buildup of coke on the surfaces of the catalyst. Regeneration of the catalyst may be accomplished by burning off the coke in a regenerator. In some embodiments, the method includes the step of regenerating the catalyst. In some embodiments, the catalyst is regenerated by exposing the catalyst to high temperature and an oxygen source in a regenerator. In some embodiments, the oxygen source is air. The regeneration of the catalyst is an oxidative and exothermic reaction.

Because the plastic pyrolysis process is endothermic, heat must be added to the reactor stages to maintain a sufficiently high temperature. The catalyst leaves the regenerator at a very high temperature. By adjusting the rate of regenerated catalyst being fed back into the reactor stages, the temperature within the reactor stages can be controlled. In some embodiments, the regenerated catalyst is only fed into the first reactor stage. In other embodiments, the regenerated catalyst is fed into each reactor stage. In some embodiments, the feed rate of regenerated is adjustable based on a predetermine target temperature. For example, if the temperature inside a particular stage exceeds an upper limit temperature, the feed rate of hot catalyst to that stage can be reduced. And if the temperature in a particular reactor stage falls below a lower limit temperature, the feed rate of hot catalyst to that reactor stage can be increased.

In some embodiments, the first and second hydrocarbon products comprise C₁-C₁₂ saturated hydrocarbons, C₁-C₁₂ unsaturated hydrocarbons, or a mixture of any two or more thereof. The products derived from one reactor stage may be the same or different than those derived in another reactor stage. In some embodiments, the hydrocarbon products include olefins, aromatic compounds, or a mixture of any two or more thereof.

The product streams from the method are collected for further processing and refinement. It is preferable that the product vapors are removed immediately upon formation within the reactor stage. In some embodiments, the separation of the product streams from the other material within the reactor stage begins within the reactor stage itself. This may be accomplished through the use of a confiner within the reactor stage, as a non-limiting example. In some embodiments using two reactor stages, the first and second product streams are collected in a separation vessel. In some embodiments using two reactor stages, the first and second product streams are combined and passed to a single set of cyclone separators. In some embodiments, the plastics pyrolysis process is integrated with a refinery, which can receive the recovered product streams and purify them in the existing FCC gas plant or further process them via hydroprocessing or catalytic cracking to produce transportation fuels or petrochemicals. Hydroprocessing may include fixed bed or ebullated bed hydrotreating or hydrocracking. Catalytic cracking may include fluid catalytic cracking (FCC), Deep Catalytic Cracking (DCC), and High Severity Fluid Catalytic Cracking (HSFCC).

In another embodiment of the invention, the plastics pyrolysis process is integrated with a petrochemical plant, which can receive the recovered products and further convert them by a gas or liquid steam cracking process to increase the production of petrochemicals, such as ethylene, propylene, butene and butadiene.

The present invention, thus generally described, will be understood more readily by reference to the following examples, which are provided by way of illustration and are not intended to be limiting of the present invention.

### EXAMPLES

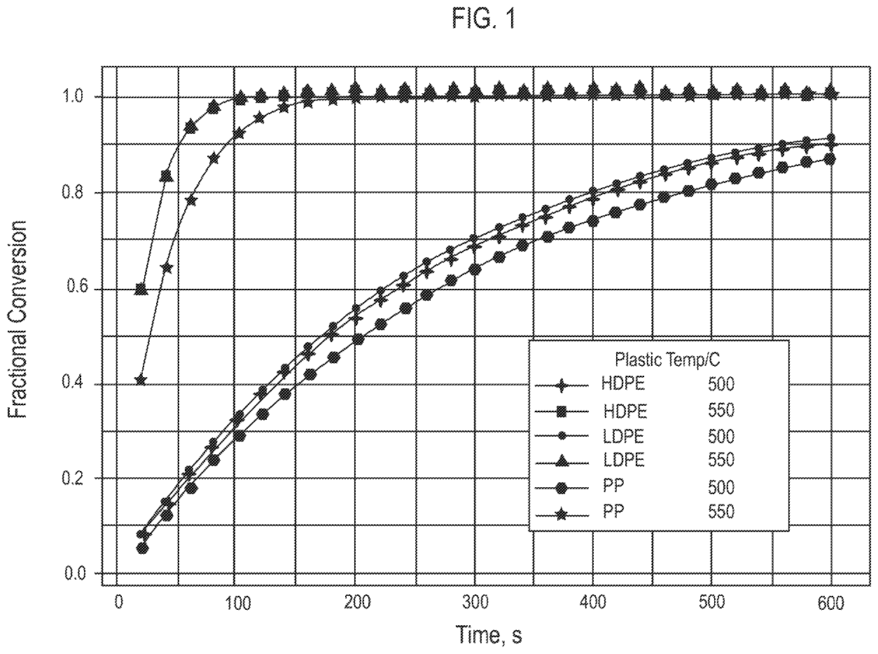

Example 1. The conversion of HDPE, LDPE, and PP at 500° C. and 550° C. are shown in FIG. 1. At 500° C., the reaction does not reach 100% conversion, even after 600 seconds. At 550° C., the reaction reaches >99% conversion in 120 s for HDPE and LDPE, and 180 seconds for PP. The reactions rates used in the calculations were experimentally determined.

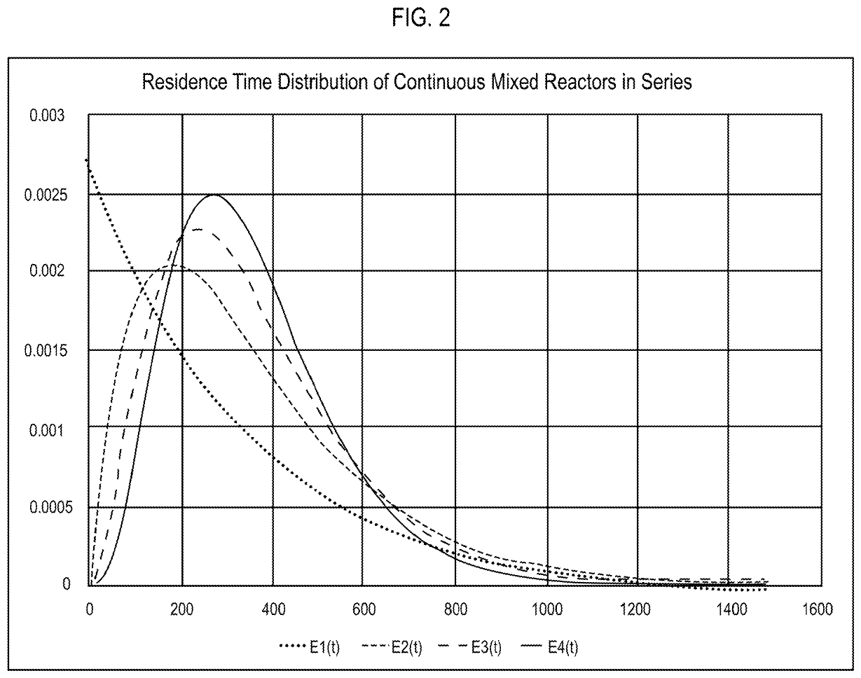

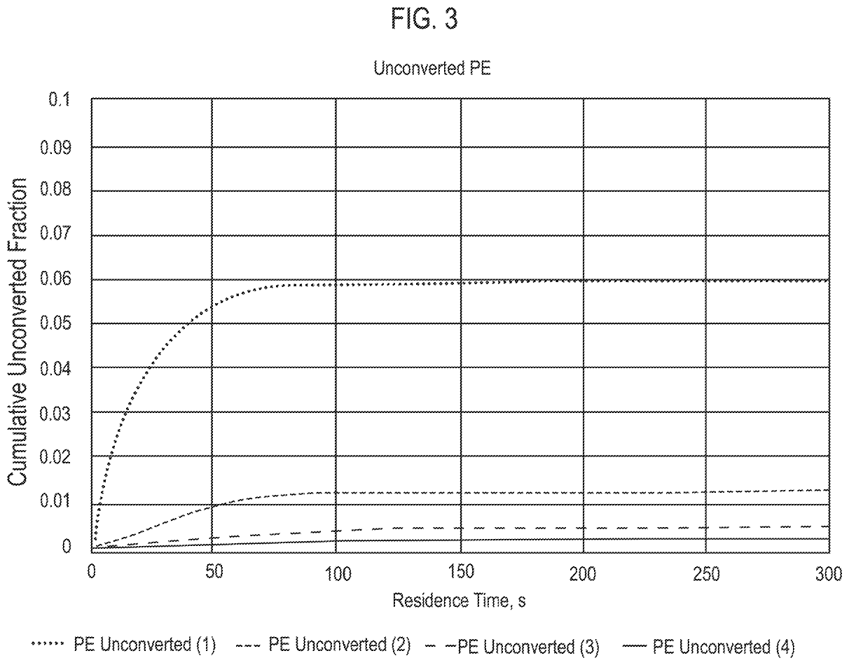

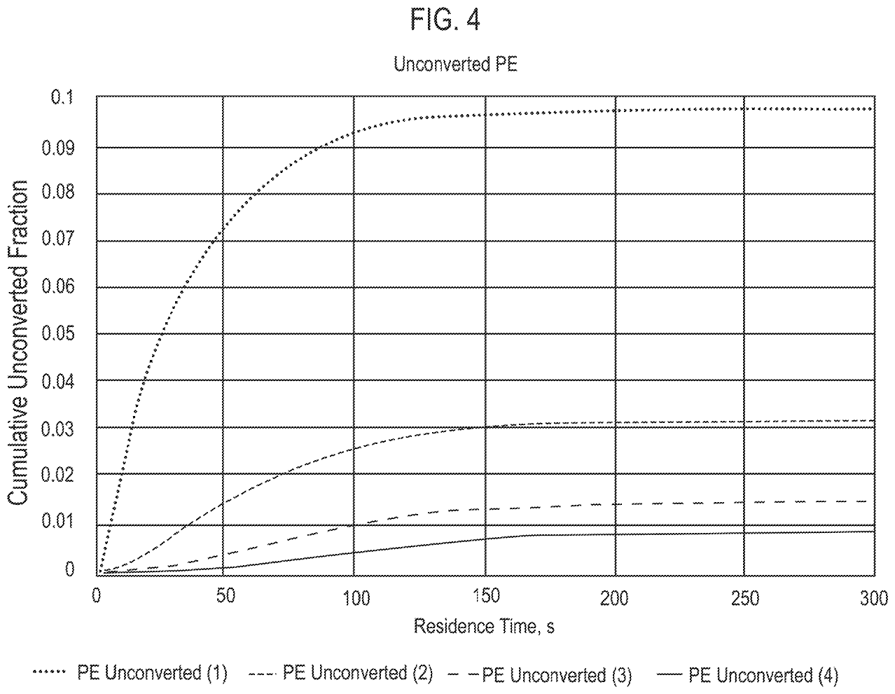

Example 2. The calculated residence time distributions for 1, 2, 3, and 4 reactors in series are shown in FIG. 2. In all four cases, the average residence time is 360 seconds. With only one reactor, there is a significant portion of plastics that exit the reactor in less than 180 seconds, before the pyrolysis reaction is complete. There is even some material that will exit the reactor immediately upon entering. This problem is eliminated by using two or more reactors in series. With increasing number of reactors in series, the residence time distribution becomes narrower and the fraction of unconverted plastics exiting the reactor is reduced. The reaction kinetics of FIG. 1 can be combined with the residence time distributions of FIG. 2 to determine the fraction of unconverted plastics in each residence time increment. The total fraction of unconverted plastics can be determined by integrating over the entire residence time distribution. The results are shown in FIGS. 3 and 4 for HDPE and PP at a reaction temperature of 550° C. For a single reactor, the unconverted PE and PP represent 6.0% and 9.8%, respectively, of the total plastics. If the unconverted plastics, entrained in the catalyst, is circulated to the regenerator, it would increase the coke yield and decrease the yield of valuable products. The extra coke would also increase the regenerator temperature, causing a decrease in catalyst circulation rate for heat-balanced units, which could further decrease conversion. Furthermore, for units not having sufficient catalyst cooling capability, the regenerator temperature may reach the metallurgical limit of vessel, necessitating a decrease in the feed throughput.

For two reactors in series, the unconverted PE and PP rapidly decrease to 1.3% and 3.2%, respectively. The unconverted PE and PP further decrease for three reactors in series to 0.42% and 1.5%, respectively, and for four reactors in series to 0.17% and 0.86%, respectively. This example clearly illustrates the benefit of using two or more reactors in series for plastics pyrolysis.

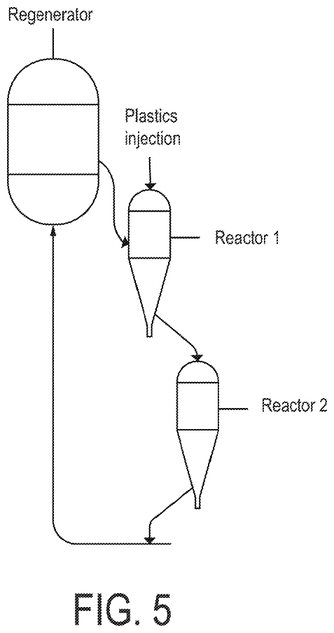

Example 3. Reactors in series can be accomplished by having separate reactor vessels or a single vessel with multiple chambers or compartments. A schematic diagram of two spouted bed reactor vessels is shown in FIG. 5. Plastics are fed to Reactor 1, together with hot catalyst from the regenerator. Reaction products are removed from Reactor 1. Spent catalyst and unreacted plastics flow from Reactor 1 to Reactor 2. Additional hot catalyst from the regenerator can be added to Reactor 2 to control the reaction temperature. Gases for fluidization or spouting are supplied to each vessel separately. The fraction of unreacted plastics in Reactor is very low, as shown in FIGS. 3 and 4. Spent catalyst from Reactor 2 is pneumatically conveyed to the regenerator. Reaction products from Reactor 2 is combined with the product from Reactor 1 and sent to product recovery and purification.

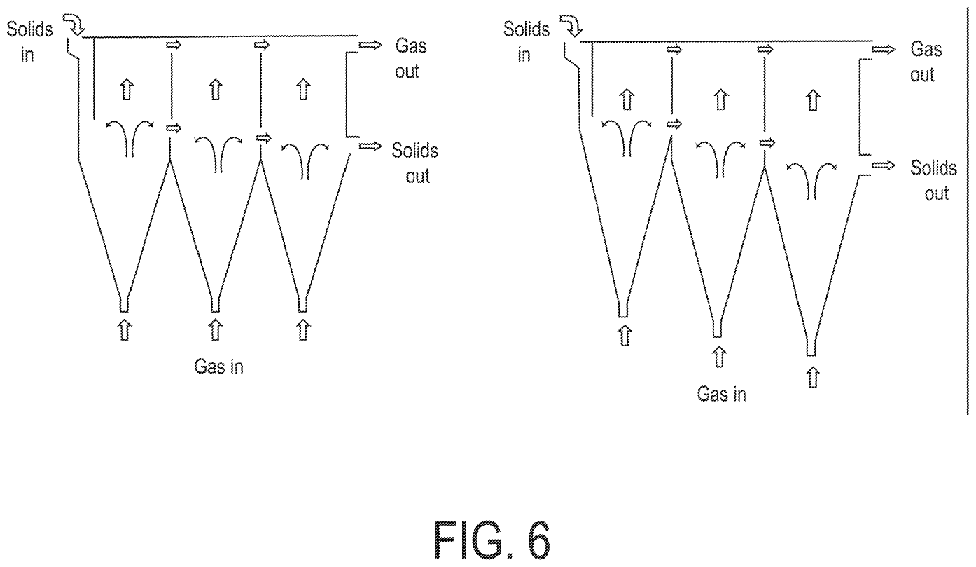

Example 4. Two arrangements of a single vessel containing three interconnected spouted bed reaction chambers are shown in FIG. 6. For ease of construction, the chambers may be in the shape of inverted pyramids, with straight sides. In the first arrangement, the chambers are positioned at the same elevation; and a weir plate is installed in the last chamber to control the level of the catalyst. Catalyst flows from one chamber to the next and over the weir plate. In this arrangement, the level of the catalyst bed and hence the amount of catalyst decreases from the first to the last chamber. In the second arrangement, the chambers are staggered so that each chamber is at a lower elevation than the preceding one, allowing catalyst to flow by gravity. In this arrangement, the amount of catalyst can be the same in each chamber.

For both arrangements, gases for fluidization or spouting are supplied separately to each chamber. Plastics are fed to the first chamber. Catalyst and unconverted plastic flows from one chamber to the next through an opening, which could be at the level of the catalyst bed, below the level of the catalyst bed or on the side of the catalyst bed. The chambers may be separated by baffles either below, above or both below and above, the surface of the catalyst bed. Hot regenerated catalyst may be directed all to the first chamber or distributed to all the chambers to control the temperature profile. The product vapors are collected from each chamber, combined downstream, and sent to product recovery and purification.
Para. 1. A system for converting plastic into lower molecular weight products, the system comprising:
   a catalyst regenerator;
   a feeder containing plastic feedstock;
   a first conical spouted bed reactor stage in fluid communication with the catalyst regenerator and in fluid communication with the feeder; and
   a second conical spouted bed reactor stage in fluid communication with the first conical spouted bed reactor stage.
Para. 2. The system of Para. 1 further comprising:
   a first reactor vessel containing the first conical spouted bed reactor stage; and
   a second reactor vessel containing the second conical spouted bed reactor stage;
   wherein the first reactor vessel and second reactor vessel are fluidly connected with at least one pipe configured to channel a flow of catalyst and unreacted plastic feedstock from the first reactor vessel to the second reactor vessel.
Para. 3. The system of Para. 2, wherein the second reactor vessel is at a lower elevation than the first reactor vessel.
Para. 4. The system of Para. 2, wherein the pipe is aerated such that the flow of catalyst and unreacted plastic feedstock from the first reactor vessel to the second reactor vessel is pneumatically driven.
Para. 5. The system of any one of Paras. 1-4, wherein the first conical spouted bed reactor stage and the second conical spouted bed reactor stage are contained in a single reactor vessel, and the first conical spouted bed reactor stage and the second conical spouted bed reactor stage are at least partially separated by baffles.
Para. 6. The system of Para. 5, wherein the baffles define at least one opening between the first conical spouted bed reactor stage and the second conical spouted bed reactor stage at the top, bottom, or at least one side of the first conical spouted bed reactor stage.
Para. 7. The system of Para. 5, wherein the first conical spouted bed reactor stage and second conical spouted bed reactor stage are at different relative elevations.
Para. 8. The system of any one of Paras. 1-7, wherein the first conical spouted bed reactor stage is configured to receive catalyst from the catalyst regenerator.
Para. 9. The system of any one of Paras. 1-8, wherein the second conical spouted bed reactor stage is in fluid communication with the catalyst regenerator and is configured to receive catalyst from the catalyst regenerator.
Para. 10. The system of Para. 8, wherein a flow of catalyst from the catalyst regenerator to the first conical spouted bed reactor stage is adjustable in response to a temperature in the first conical spouted bed reactor stage falling below a predetermined temperature set point.
Para. 11. The system of any one of Paras. 1-10, wherein the first conical spouted bed reactor stage is operated in a pyrolysis regime.
Para. 12. The system of Para. 9, wherein a flow of catalyst from the catalyst regenerator to the second conical spouted bed reactor stage is adjustable in response to a temperature in the second conical spouted bed reactor stage falling below a predetermined temperature set point.
Para. 13. The system of any one of Paras. 1-12 further comprising a draft tube extending from the bottom of the first conical spouted bed reactor stage toward the top of the first conical spouted bed reactor stage, the draft tube comprising a cylindrical tube having an outer diameter smaller than the inner diameter of the bottom of the first conical spouted bed reactor stage and at least one opening extending upward from the bottom of the draft tube.
Para. 14. The system of any one of Paras. 1-13 further comprising a confiner extending from the top of the first conical spouted bed reactor stage toward the bottom of the first conical spouted bed reactor stage, the confiner comprising a cylindrical tube having an outer diameter smaller than the inner diameter of the top of the first conical spouted bed reactor stage.
Para. 15. The system of any one of Paras. 1-14 further comprising a third conical spouted bed reactor stage in fluid communication with the second conical spouted bed reactor stage.
Para. 16. The system of any one of Paras. 1-15, wherein, in operation, the first conical spouted bed reactor stage has a temperature of about 300° C. to about 650° C., or from about 450° C. to about 600° C., or from about 480° C. to about 550° C.
Para. 17. The system of any one of Paras. 1-16, wherein, in operation, the second conical spouted bed reactor stage has a temperature from about 300° C. to about 650° C., or from about 450° C. to about 600° C., or from about 480° C. to about 550° C.
Para. 18. The system of any one of Paras. 1-17 further comprising a gas feed system in fluid communication with the first conical spouted bed reactor stage and the second conical spouted bed reactor stage, the gas feed system being configured to feed a motive gas to the first conical spouted bed reactor stage and the second conical spouted bed reactor stage.
Para. 19. The system of Para. 18 wherein the motive gas contains less than 1.0 wt. % oxygen or, more preferably, less than 0.1 wt. % oxygen.
Para. 20. The system of any one of Paras. 1-19 further comprising a set of separation cyclones in fluid communication with the first conical spouted bed reactor stage and the second conical spouted bed reactor stage.
Para. 21. A method of producing hydrocarbon product from plastic, the method comprising:
   feeding a plastic feedstock and motive gas into a first conical spouted bed reactor stage containing a catalyst to produce a first product vapor and a first residual plastic;
   separating at least a portion of the first product vapor from the motive gas and the first residual plastic to produce a first product stream comprising the first product vapor;
   feeding the first residual plastic from the first conical spouted bed reactor stage into a second conical spouted bed reactor stage containing a catalyst to produce a second product vapor and a second residual plastic; and
   separating at least a portion of the second product vapor from the motive gas and the second residual plastic to produce a second product stream comprising the second product vapor.
Para. 22. The method of Para. 21, further comprising transferring at least a portion of the catalyst from the first conical spouted bed reactor stage to the second conical spouted bed reactor stage.
Para. 23. The method of any one of Paras. 21-22 further comprising transferring at least of portion of the catalyst from the second conical spouted bed reactor stage to a regenerator.
Para. 24. The method of any one of Paras. 21-23 further comprising feeding catalyst from the regenerator into the first conical spouted bed reactor stage.
Para. 25. The method of Para. 23 further comprising feeding catalyst from the regenerator into the second conical spouted bed reactor stage.
Para. 26. The method of any one of Paras. 22-25, wherein the transfer of the portion of the catalyst from the first conical spouted bed reactor stage to the second conical spouted bed reactor stage is at least partly driven by a flow of motive gas.
Para. 27. The method of any one of Paras. 23-26, wherein the transfer of the portion of the catalyst from the second conical spouted bed reactor stage to the regenerator is at least partly driven by a flow of motive gas.
Para. 28. The method of any one of Paras. 21-27, wherein the first conical spouted bed reactor stage has a temperature from about 300° C. to about 650° C., or from about 450° C. to about 600° C., or from about 480° C. to about 550° C.
Para. 29. The method of Para. 28, wherein the temperature of the first conical spouted bed reactor stage is controlled in part through feeding hot catalyst into the first conical spouted bed reactor stage from the regenerator.
Para. 30. The method of any one of Paras. 21-29, wherein the second conical spouted bed reactor stage has a temperature from about 300° C. to about 650° C., or from about 450° C. to about 600° C., or from about 480° C. to about 550° C.
Para. 31. The method of Para. 29, wherein the temperature of the second conical spouted bed reactor stage is controlled in part through feeding hot catalyst into the second conical spouted bed reactor stage from the regenerator.
Para. 32. The method of any one of Paras. 21-31, wherein the plastic feedstock is first shredded to a nominal size of about 1 mm to about 20 mm, or about 8 mm to about 10 mm, prior to feeding into the first conical spouted bed reactor stage.
Para. 33. The method of any one of Paras. 21-31, wherein the first conical spouted bed reactor stage and the second conical spouted bed reactor stage are both contained within a single reactor vessel.
Para. 34. The method of any one of Paras. 21-31, further comprising feeding the second residual plastic from the second conical spouted bed reactor stage into a third conical spouted bed reactor stage containing catalyst to produce a third product vapor and a residue; and separating the third product vapor, motive gas, and residue to produce a third product stream comprising the third product vapor.
Para. 35. The method of any one of Paras. 21-34 further comprising directing the first product stream and the second product stream into a cyclone separator.
Para. 36. The method of Para. 35, wherein the first product stream and second product stream are combined before being directed into a set of cyclone separators.
Para. 37. The method of any one of Paras. 21-36 further comprising collecting the first product stream and the second product stream into a separation vessel.
Para. 38. The method of any one of Paras. 21-37, wherein the plastic feedstock comprises high density polyethylene, medium density polyethylene, low density polyethylene, polypropylene, polyethylene terephthalate, polyvinyl chloride, or a mixture of any two or more thereof.
Para. 39. The method of any one of Paras. 21-38, wherein the first and second hydrocarbon products comprise C₁-C₁₂ saturated hydrocarbons, C₁-C₁₂ unsaturated hydrocarbons, or a mixture of any two or more thereof, and wherein the first and second hydrocarbon products may be the same or different.
Para. 40. The method of any one of Paras. 21-39, wherein the hydrocarbon product comprises olefins, aromatic compounds, or a mixture of any two or more thereof.
Para. 41. The method of any one of Paras. 21-40 further comprising processing and refining one or more of the first hydrocarbon product, the second hydrocarbon product, the first plastic residue, or the second plastic residue in a steam cracker, a hydrocracker, a fluid catalytic cracker, a deep catalytic cracker, a high severity fluid catalytic cracker, a steam reformer, a liquid cracker gas plant, or aromatic recovery unit.
Para. 42. The method of any one of Paras. 21-41, wherein the size of the first conical spouted bed reactor stage is the same as the size of the second conical spouted bed reactor stage.
Para. 43. The method of any one of Paras. 21-42, wherein the method is performed continuously.
Para. 44. The method of any one of Paras. 21-43, wherein the plastic feedstock comprises a waste plastic.
Para. 45. The method of any one of Paras. 21-44, wherein separating the at least a portion of the first product vapor from the motive gas and the first residual plastic to produce a first product stream comprising the first product vapor occurs within the first conical spouted bed reactor stage.
Para. 46. The method of any one of Paras. 21-45, wherein the first product stream is removed from the first conical spouted bed reactor stage immediately as it is formed.
Para. 47. The method of any one of Paras. 21-46, wherein separating the at least a portion of the second product vapor from the motive gas and the second residual plastic to produce a second product stream comprising the second product vapor occurs within the second conical spouted bed reactor stage.
Para. 48. The method of any one of Paras. 21-47, wherein the second product stream is removed from the second conical spouted bed reactor stage immediately as it is formed.
Para. 49. The method of any one of Paras. 21-48, wherein the average gas phase residence time in the first conical spouted bed reactor stage is about 0.2 seconds to about 60 seconds, or preferably about 0.5 seconds to about 5 seconds.
Para. 50. The method of any one of Paras. 21-49, wherein the average gas phase residence time in the second conical spouted bed reactor stage is about 0.2 seconds to about 60 seconds, or preferably about 0.5 seconds to about 5 seconds.
Para. 51. The method of any one of Paras. 21-50, wherein the motive gas contains less than 1.0 wt. % oxygen or, more preferably, less than 0.1 wt. % oxygen.
Para. 52. The method of any one of Paras. 21-51, wherein the first conical spouted bed reactor stage and the first conical spouted bed reactor stage are operated in a fast pyrolysis regime.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions, or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A system for converting plastic into lower molecular weight products, the system comprising:
a catalyst regenerator;
a feeder containing plastic feedstock;
a first conical spouted bed reactor stage in fluid communication with the catalyst regenerator and in fluid communication with the feeder; and
a second conical spouted bed reactor stage in fluid communication with the first conical spouted bed reactor stage.

2. The system of claim 1 further comprising:
a first reactor vessel containing the first conical spouted bed reactor stage; and
a second reactor vessel containing the second conical spouted bed reactor stage;
wherein the first reactor vessel and second reactor vessel are fluidly connected with at least one pipe configured to channel a flow of catalyst and unreacted plastic feedstock from the first reactor vessel to the second reactor vessel.

3. The system of claim 2, wherein the second reactor vessel is at a lower elevation than the first reactor vessel, and/or wherein the pipe is aerated such that the flow of catalyst and unreacted plastic feedstock from the first reactor vessel to the second reactor vessel is pneumatically driven.

4. The system of claim 1, wherein the first conical spouted bed reactor stage and the second conical spouted bed reactor stage are contained in a single reactor vessel, and the first conical spouted bed reactor stage and the second conical spouted bed reactor stage are at least partially separated by baffles.

5. The system of claim 4, wherein the baffles define at least one opening between the first conical spouted bed reactor stage and the second conical spouted bed reactor stage at the top, bottom, or at least one side of the first conical spouted bed reactor stage, and/or wherein the first conical spouted bed reactor stage and second conical spouted bed reactor stage are at different relative elevations.

6. The system of claim 1, wherein the first conical spouted bed reactor stage is configured to receive catalyst from the catalyst regenerator, and/or wherein the second conical spouted bed reactor stage is in fluid communication with the catalyst regenerator and is configured to receive catalyst from the catalyst regenerator, and/or wherein the first conical spouted bed reactor stage is operated in a pyrolysis regime.

7. The system of claim 6, wherein a flow of catalyst from the catalyst regenerator to the first conical spouted bed reactor stage is adjustable in response to a temperature in the first conical spouted bed reactor stage falling below a predetermined temperature set point, and/or wherein a flow of catalyst from the catalyst regenerator to the second conical spouted bed reactor stage is adjustable in response to a temperature in the second conical spouted bed reactor stage falling below a predetermined temperature set point.

8. The system of claim 1 further comprising one or more of the following:
a draft tube extending from the bottom of the first conical spouted bed reactor stage toward the top of the first conical spouted bed reactor stage, the draft tube comprising a cylindrical tube having an outer diameter smaller than the inner diameter of the bottom of the first conical spouted bed reactor stage and at least one opening extending upward from the bottom of the draft tube;
a confiner extending from the top of the first conical spouted bed reactor stage toward the bottom of the first conical spouted bed reactor stage, the confiner comprising a cylindrical tube having an outer diameter smaller than the inner diameter of the top of the first conical spouted bed reactor stage; and
a third conical spouted bed reactor stage in fluid communication with the second conical spouted bed reactor stage.

9-10. (canceled)

11. The system of claim 1, wherein, in operation, the first conical spouted bed reactor stage has a temperature of about 300° C. to about 650° C., and/or wherein, in operation, the second conical spouted bed reactor stage has a temperature from about 300° C. to about 650° C.

12. The system of claim 1 further comprising a gas feed system in fluid communication with the first conical spouted bed reactor stage and the second conical spouted bed reactor stage, the gas feed system being configured to feed a motive gas to the first conical spouted bed reactor stage and the second conical spouted bed reactor stage.

13. The system of claim 12 wherein the motive gas contains less than 1.0 wt. % oxygen or, more preferably, less than 0.1 wt. % oxygen.

14. The system of claim 1 further comprising a set of separation cyclones in fluid communication with the first conical spouted bed reactor stage and the second conical spouted bed reactor stage.

15. A method of producing hydrocarbon product from plastic, the method comprising:
feeding a plastic feedstock and motive gas into a first conical spouted bed reactor stage containing a catalyst to produce a first product vapor and a first residual plastic;
separating at least a portion of the first product vapor from the motive gas and the first residual plastic to produce a first product stream comprising the first product vapor;
feeding the first residual plastic from the first conical spouted bed reactor stage into a second conical spouted bed reactor stage containing a catalyst to produce a second product vapor and a second residual plastic; and
separating at least a portion of the second product vapor from the motive gas and the second residual plastic to produce a second product stream comprising the second product vapor.

16. The method of claim 15, further comprising transferring at least a portion of the catalyst from the first conical spouted bed reactor stage to the second conical spouted bed reactor stage, and/or further comprising transferring at least of portion of the catalyst from the second conical spouted bed reactor stage to a regenerator.

17. (canceled)

18. The method of claim 15, wherein the transfer of the portion of the catalyst from the first conical spouted bed reactor stage to the second conical spouted bed reactor stage is at least partly driven by a flow of motive gas, and/or wherein the transfer of the portion of the catalyst from the second conical spouted bed reactor stage to the regenerator is at least partly driven by a flow of motive gas.

19. (canceled)

20. The method of claim 18, wherein the temperature of the first conical spouted bed reactor stage is controlled in part through feeding hot catalyst into the first conical spouted bed reactor stage from the regenerator, and/or the temperature of the second conical spouted bed reactor stage is controlled in part through feeding hot catalyst into the second conical spouted bed reactor stage from the regenerator.

21. The method of claim 15, wherein
the plastic feedstock is first shredded to a nominal size of about 1 mm to about 20 mm, or about 8 mm to about 10 mm, prior to feeding into the first conical spouted bed reactor stage.

22-23. (canceled)

24. The method of claim 15 further comprising directing the first product stream and the second product stream into a cyclone separator.

25. The method of claim 24, wherein the first product stream and second product stream are combined before being directed into a set of cyclone separators.

26. (canceled)

27. The method of claim 15, wherein one or more of the following:
the plastic feedstock comprises high density polyethylene, medium density polyethylene, low density polyethylene, polypropylene, polyethylene terephthalate, polyvinyl chloride, or a mixture of any two or more thereof;
the first and second hydrocarbon products comprise C₁-C₁₂ saturated hydrocarbons, C₁-C₁₂ unsaturated hydrocarbons, or a mixture of any two or more thereof, and wherein the first and second hydrocarbon products may be the same or different, and/or wherein the first and second hydrocarbon products comprise olefins, aromatic compounds, or a mixture of any two or more thereof;
the method further comprises processing and refining one or more of the first hydrocarbon product, the second hydrocarbon product, the first plastic residue, or the second plastic residue in a steam cracker, a hydrocracker, a fluid catalytic cracker, a deep catalytic cracker, a high severity fluid catalytic cracker, a steam reformer, a liquid cracker gas plant, or aromatic recovery unit;
the size of the first conical spouted bed reactor stage is the same as the size of the second conical spouted bed reactor stage, and/or wherein the method is performed continuously, and/or wherein the plastic feedstock comprises a waste plastic;
separating the at least a portion of the first product vapor from the motive gas and the first residual plastic to produce a first product stream comprising the first product vapor occurs within the first conical spouted bed reactor stage;
the first product stream is removed from the first conical spouted bed reactor stage immediately as it is formed, and/or wherein the second product stream is removed from the second conical spouted bed reactor stage immediately as it is formed;
separating the at least a portion of the second product vapor from the motive gas and the second residual plastic to produce a second product stream comprising the second product vapor occurs within the second conical spouted bed reactor stage;
the average gas phase residence time in the first conical spouted bed reactor stage is about 0.2 seconds to about 60 seconds, or preferably about 0.5 seconds to about 5 seconds, and/or wherein the average gas phase residence time in the second conical spouted bed reactor stage is about 0.2 seconds to about 60 seconds, or preferably about 0.5 seconds to about 5 seconds, and
motive gas contains less than 1.0 wt. % oxygen or, more preferably, less than 0.1 wt. % oxygen, and/or wherein the first conical spouted bed reactor stage and the first conical spouted bed reactor stage are operated in a fast pyrolysis regime.

28-35. (canceled)
